(19) United States
(12) Patent Application Publication
(10) Pub. No.: US 2025/0041111 A1
(43) Pub. Date: Feb. 6, 2025

(54) ROBOTIC AUTO EYE LUBRICATOR

(71) Applicant: Alcon Inc., Fribourg (CH)

(72) Inventors: Patrick TERRY, Goleta, CA (US); Sumit PALIWAL, Irvine, CA (US); Mark Andrew ZIELKE, Lake Forest, CA (US); Lance NOLLER, Lake Forest, CA (US); Nanhong LOU, Southlake, TX (US)

(21) Appl. No.: 18/790,802

(22) Filed: Jul. 31, 2024

Related U.S. Application Data
(60) Provisional application No. 63/517,234, filed on Aug. 2, 2023.

(51) Int. Cl. A61F 9/00; A61B 3/10; A61B 34/20; A61B 34/30; A61B 90/20

(52) U.S. Cl. CPC A61F 9/0026; A61B 3/102; A61B 2034/2055; A61B 34/30; A61B 90/20

(57) ABSTRACT

A system and method for assisting a surgeon performing an ophthalmic procedure on an eye of a patient, the system including a lubrication assembly. The lubrication assembly may be disposed on a proximal end of a robotic arm, a proximal end of a surgical microscope, or a proximal end of a mobile stand or frame. Dispersal of lubricant from the lubrication assembly may be upon receipt of a direct input from a surgeon, according to a predetermined schedule, or according to an image processing protocol. The lubrication assembly may be moved into position over the eye of the patient manually by the surgeon or through the indirect command of a motion controller which is operable to move the robotic arm or the surgical microscope so as to extend the lubrication assembly into a desired position.

### CROSS-REFERENCE TO RELATED APPLICATIONS

This application claims benefit of and priority to U.S. Provisional Patent Application No. 63/517,234, filed on Aug. 2, 2023. The entire contents of this application are incorporated herein by reference.

### INTRODUCTION

Ophthalmic or ocular surgeries require frequent lubrication while being performed. The eye of the patient must be constantly lubricated during all types of ophthalmic procedures so as to avoid damage to ocular tissue. Traditionally, while the surgeon is performing the ophthalmic procedure, a nurse or other medical professional is within the vicinity with a dropper or syringe filled with lubricant to manually disperse the lubricant. When the surgeon calls out for more lubricant, the nurse then manually applies it to the patient's eye using the dropper.

However, manually applying lubricant to a patient's eye carries with it several disadvantages. For example, as the lubricant is being applied to the patient's eye, this can temporarily block or obstruct the surgeon's view of the patient, leading to inefficient performance of the ophthalmic procedure. Additionally, the nurse applying the lubricant may be applying either too much, too little, or may be applying lubricant to the wrong portion of the patient's eye, thereby leading to inefficient use of a lubricant, which can be expensive and therefore increase the overall cost of the ophthalmic procedure. Furthermore, because the assistant or surgeon must constantly judge the lubrication state of the patient's eye, this can lead to too much elapsed time between lubrication applications, which can make the ophthalmic procedure more uncomfortable for the patient than is necessary. Alternatively, if too much lubrication is applied, this can cause excessive runoff and obscure the surgeon's view at critical points during a surgical procedure.

### SUMMARY

A lubrication assembly for lubricating an eye of a patient during an ophthalmic procedure is provided. In certain embodiments, the lubrication assembly comprises a mounting bar, an output coupled to the mounting bar, means for disposing the output into a desired position relative to the eye of the patient coupled to the mounting bar, a lubrication source fluidly coupled to the output, a cable communicating a lubricant between the lubrication source and the output, and a camera coupled to the mounting bar.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 is a perspective view illustrating a surgery being performed in an operating environment with a surgical system, according to aspects of this disclosure.

FIG. 2 is a perspective view of a robotic arm for use with the surgical system of FIG. 1, according to certain aspects of this disclosure.

FIG. 3A is a magnified frontal view depicting a lubrication assembly at a working end of the robotic arm of FIG. 2, according to certain aspects of the disclosure.

FIG. 3B is an exemplary illustration of a corneal-cross link add-on which may be selectively disposed on the lubrication assembly of FIG. 3A, according to certain aspects of the disclosure.

FIG. 3C is an exemplary illustration of an OCT add-on which may be selectively disposed on the lubrication assembly of FIG. 3A, according to certain aspects of the disclosure.

FIG. 4 is a block diagram schematically illustrating components of the surgical system, according to certain aspects of the disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Various examples will now be described more fully with reference to the accompanying drawings. Systems like those disclosed here may, however, be embodied in various different forms and should not be construed as limited to the examples set forth here.

As described herein, the term “lubricant” generally refers to ocular lubricants, including but not limited to eye drops with preservatives, preservative-free eye drops, other ophthalmic liquids, gels, or ointments, and the like.

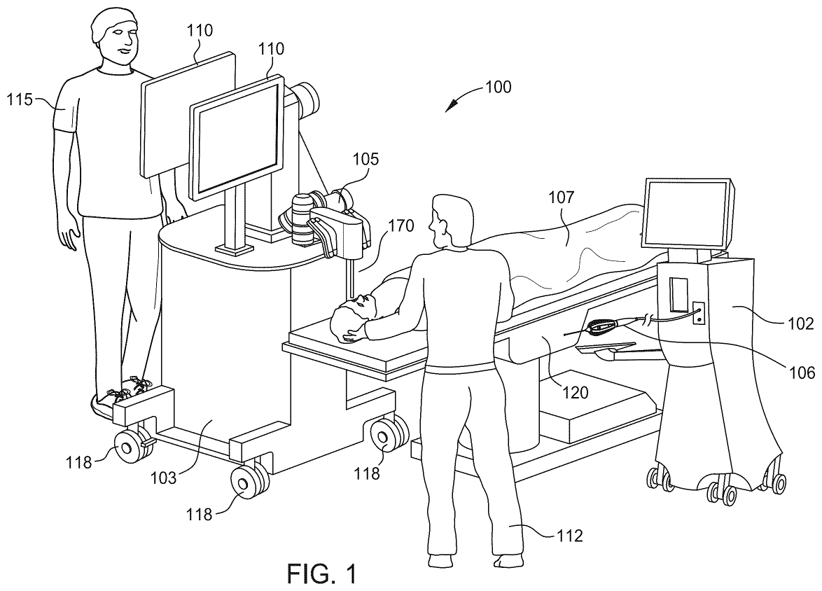

FIG. 1 depicts a system 100 for performing an ophthalmic surgery. The system 100 includes a surgical console 102 and a robotic arm 105. The surgical console 102 may include, or be operably coupled to (e.g., physically or wirelessly), one or more modules, systems, devices, and/or surgical tools for performing one or more surgical procedures. For example, in certain embodiments, the surgical console 102 comprises one or more ports for coupling a surgical tool 106 to an internal fluid source, vacuum source, and/or driver. In certain embodiments, the surgical console 102 may be in physical or wireless communication with the robotic arm 105.

The robotic arm 105 is operable to hold, move, and insert a lubrication assembly 170 into an operating theater and within a close proximity to an eye of a medical patient 107. Although it is currently contemplated that the medical patient will most frequently be a human patient, in veterinary applications the “patient” can be a non-human animal, and the term as it appears in this disclosure should be read to encompass either possibility. Additionally, while a robotic arm comprising a series of components is shown and described, in certain other embodiments, a robotic holding platform or other apparatus can be used. For example, rather than a serial robotic arm, a series or plurality of cascaded linear rails may be utilized to secure, move, and provide lubrication assembly 170 to an operating theater and within close proximity to the eye of the patient 107.

The robotic arm 105 may be supported by any suitable device or system within an operating environment. In the example of FIG. 1, the robotic arm 105 is supported by a cart 103. The cart 103 can itself be fixed or movable with respect to a structure for supporting the patient 107. FIG. 1 depicts an installation in which the cart 103 is supported on wheels 118 that allow the cart 103 to be rolled into position with respect to a patient support table 120, which is illustrated as fixed to the floor of a surgical suite in which the procedure is performed. In certain embodiments, however, the robotic arm 105 may be supported by the surgical console 102, the patient support table 120, or another supporting structure within the operating environment. In certain embodiments, the robotic arm 105 may be coupled to or incorporated into an ophthalmic surgical microscope along with the lubrication assembly 170 so that the robotic arm 105 may manipulate both the lubrication assembly 170 and the ophthalmic surgical microscope at the same time.

The system 100 may further include one or more video display monitors 110 for delivering information and images to medical personnel in the course of a surgery. FIG. 1 illustrates a system with two display monitors 110 disposed on the cart 103 for use, for example, by a surgeon 112 and an assistant 115. The display monitors 110 can be communicatively coupled with the surgical console 102, a visualization system within the operating environment, the ophthalmic surgical microscope, and/or the tool assembly supported by the robotic arm 105. In some embodiments, the display monitors 110 can receive information (e.g., surgical parameters) and/or images from the surgical console 102 and tool assembly, respectively, and display the information and images on the display monitors 110. The surgical console 102 can also send signals to the display monitors 110 for performing operations (e.g., starting and stopping video recording).

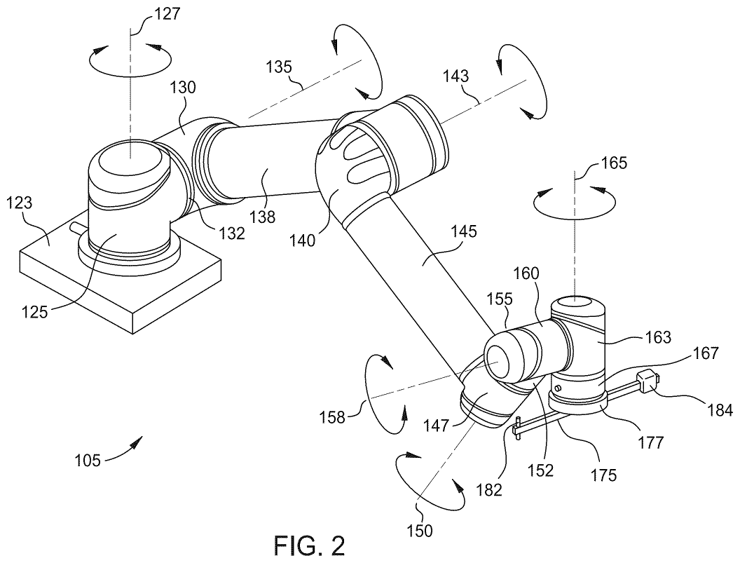

Turning now to FIG. 2, elements of the robotic arm 105 are illustrated in more detail. As shown, the robotic arm 105 includes a support base 123 which may, in certain embodiments, be configured to mount to the cart 103. Alternatively, as described above, the support base 123 may be mounted to another fixture within an operating environment, or may be a standalone component. The robotic arm further includes a first arm joint 125 that is rotatable with respect to the support base 123 around a first rotation axis 127.

A second arm joint 130 is fixed to the first arm joint 125 via a first link 132. The second arm joint 130 is rotatable with respect to the first arm joint 125 and the first link 132 around a second rotation axis 135. In certain embodiments, the second rotation axis 135 is perpendicular to the first rotation axis 127.

A second link 138 attaches a third arm joint 140 to the second arm joint 130. The third arm joint 140 can rotate with respect to the second link 138 around a third rotation axis 143. In certain embodiments, the third rotation axis 143 is parallel with the second rotation axis 135.

A third link 145 extends between the third arm joint 140 and a fourth arm joint 147. The fourth arm joint 147 rotates with respect to the third link 145 around a fourth rotation axis 150. In certain embodiments, the fourth rotation axis 150 is parallel with the second rotation axis 135.

A fourth link 152 joins the fourth arm joint 147 to a fifth arm joint 155. The fifth arm joint 147 rotates with respect to the fourth link 152 around a fifth rotation axis 158. In certain embodiments, the fifth rotation axis 158 is perpendicular to the first rotation axis 127.

A fifth link 160 attached to the fifth arm joint 155 carries a sixth arm joint 163, which rotates with respect to the fifth link 160 around a sixth axis of rotation 165. In certain embodiments, the sixth rotation axis 165 is perpendicular to the fifth rotation axis 158. The sixth arm joint 163 carries sixth link 167. In this embodiment, a working element in the form of a lubrication assembly 170 extends from a working end 172 of the sixth link, in a direction parallel to the arm's sixth axis of rotation 165.

Please note the configuration of the robotic arm 105 in FIG. 2 is only exemplary, and the exact configuration of the robotic arm 105 can vary considerably in any given embodiment. For example, in certain embodiments, though, that the robotic arm 105 include elements providing movement with at least six degrees of freedom, to facilitate effective lubrication of the patient's eye during an ophthalmic procedure. For example, a model UR5e robotic arm supplied by Universal Robots, a robotic equipment manufacturer with its headquarters in Odense, Denmark (https://www.universal-robots.com) could be incorporated into the structure of robotic arm 105, however in certain embodiments, other equivalent robotic elements or arms are used.

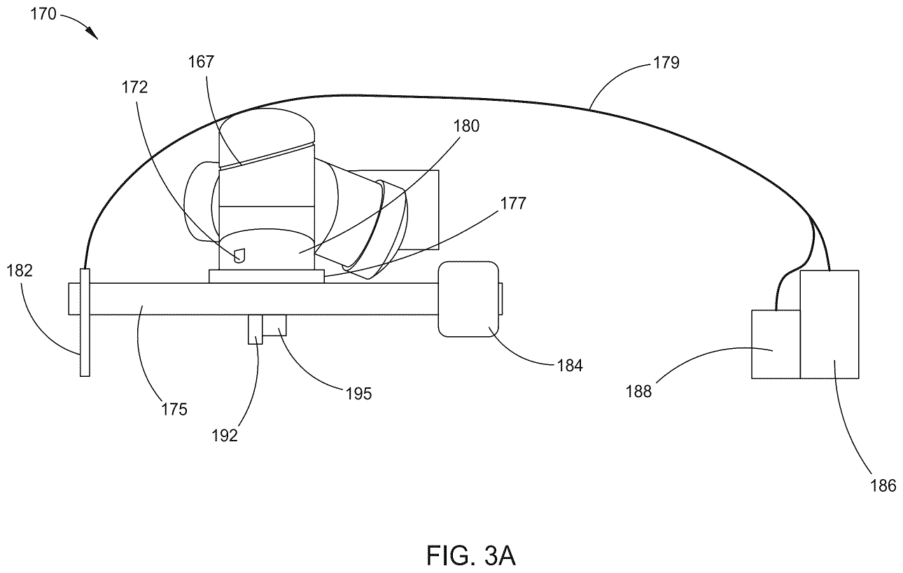

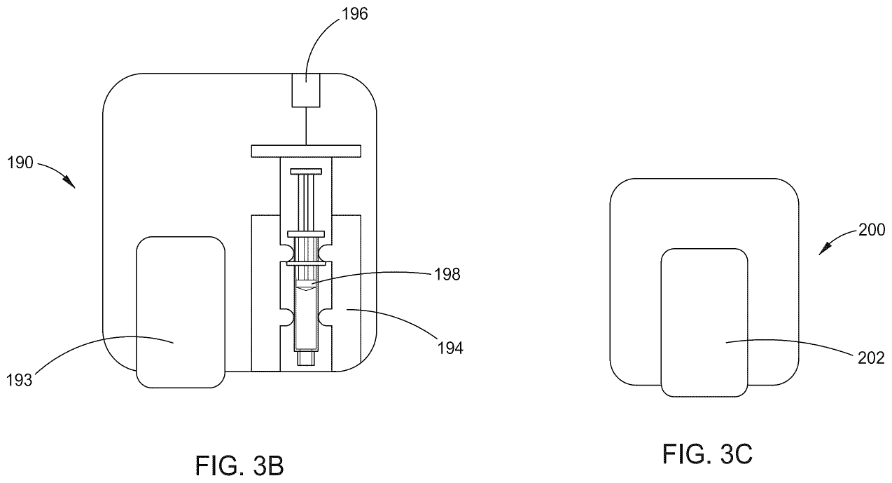

FIG. 3A depicts an exemplary lubrication assembly 170 which can be coupled at the working end 172 of the sixth robotic arm link 167. The lubrication assembly 170 includes a force/torque sensor 177 coupled to a mounting bar 175. In certain embodiments, the force/torque sensor 177 and the mounting bar 175 are fixed to the working end 172 of the sixth arm joint 167 at a proximal end 180 of the lubrication assembly 170, with an internal fluid line (not seen) disposed within the sixth arm joint 167 for communicating (e.g., flowing or providing) a lubricant to an internal volume of the lubrication element 175. In certain embodiments, one end of the mounting bar 175 comprises an output 182, while an opposing end of the mounting bar 175 comprises an add-on slot 184 which allows a plurality of different add-ons or additional optional components to be selectively added to the lubrication assembly 170. In certain embodiments, the output 182 is a needle-less or needle free syringe. In other embodiments, the output 182 may be an eye dropper, pipette, tubing, or other dispenser or nozzle configured to dispense fluid in a precise and controlled manner.

In some embodiments, a cable 179 comprising a fluid line and an electrical connection that is separate from the robotic arm 105 may be provided for supplying lubricant and/or other fluids, such as water, directly to the output 182 of the lubrication assembly 170, an opposing end of the cable 179 being communicated to a lubricant reservoir and pump sub-assembly 186, or other lubricant source. In certain embodiments, the reservoir and pump sub-assembly 186 comprises a sub-assembly controller 188 that is configured to transmit digital signals to an additional apparatus 230 of the system 100, as detailed further below. In certain embodiments, the lubrication assembly 170 comprises an electronically controlled release valve (not seen) disposed in either a distal end of the output 182, the reservoir and pump sub-assembly 186, or at any point of the cable 179 or connection elements disposed there between. In certain other embodiments, the level of lubricant within the lubrication assembly 170 may be maintained by refilling or replenishing a refillable cartridge inserted into the lubrication assembly, or by a direct injection of additional lubricant by the surgeon 112 or the assistant 115 through a self-scaling port (not seen) defined on the lubrication assembly 170 via a separate syringe. In certain embodiments, the lubricant reservoir and pump sub-assembly 186 is pressure regulated so as to provide a consistent and even flow of lubricant through the output 182.

In certain embodiments, the lubrication assembly 170 may be fixed permanently at the working end 172 of the sixth arm joint 163 or the mounting bar 175. In many cases, though, it may be preferable that the lubrication assembly 170 be conveniently removable and replaceable at working end 172 of the sixth arm joint 163 or the mounting bar 175. A lubrication assembly 170 that is conveniently removable with respect to the robotic arm 105 can allow for the replacement of a malfunctioning lubrication assembly 170, for the removal and sterilization of a reusable lubrication assembly 170 between procedures, for the removal and disposal of a single-use, disposable lubrication assembly 170 after a completed procedure, and/or for other advantages as appropriate depending on the particular application. As also seen in FIG. 3A, in certain embodiments the lubrication assembly 170 comprises an illumination clement 192 and a camera 195 coupled to or adjacent to the mounting bar 175. The illumination element 192 delivers visible light or another suitable illumination energy from the working end 172 of the robotic arm 105 to a surgery site or another location of interest. Illumination energy reflected from the target site returns to the camera 195, where it is received and passed to other elements of the system for processing and display, e.g., as still images or video for display on display monitors 110.

Specifically, digital signals corresponding to images received at the camera 195 are processed and sent to the system's display monitors 110, where they can be seen and used by the surgeon 112, the assistant 115, and others involved in the procedure. In certain embodiments the digital signals received at the camera 195 may for example be used to continuously track or monitor the relative moisture level of the patient's eye, the position of the lubrication assembly 170 relative to the patient's eye, and/or to assist in the targeted lubrication of the patient's eye as is further detailed below.

In certain embodiments, a corneal cross-link add-on 190 as seen in FIG. 3B may be coupled or slotted into the add-on slot 184 of the mounting bar 175. The corneal cross-link add-on 190 in certain embodiments comprises a UV light source 193 and a 1-Degree of Freedom (DoF) actuator 196 for actuating a syringe 198 disposed or accommodating within a snap-in clip 194. The 1-DoF actuator 196 and the UV light source 193 are connected to the electrical connection within the cable 179 which in turn is connected to the sub-assembly controller 188. The syringe 198 is fluidly coupled to the output 182 through tubing which may be external or internal to the mounting bar 175 according to certain embodiments. In some embodiments, when the 1-DoF actuator 196 is activated by the sub-assembly controller 188, liquid vitamins or another fluid is applied to the patient's eye through the output 182 according to known corneal biomechanics algorithms. The UV light source 193 may then be activated to apply UV light to the patient's eye and assist in carrying out the corneal cross-linking procedure. For example, in certain embodiments, the robotic arm 105 can be utilized to disperse vitamin B₂ or Riboflavin, either under a predetermined time schedule or based upon accurate imaging processing and corneal biomechanics algorithms. UV light from the UV light source 193 is then applied onto the patient's eye, and particularly on to the dispersed liquids, until the eye soaks in the dispersed vitamin B₂ or Riboflavin sufficiently.

In certain other embodiments, an OCT add-on 200 as seen in FIG. 3C may be coupled or slotted into the add-on slot 184 of the mounting bar 175. The OCT add-on 200 in certain embodiments comprises an optical coherence tomography (OCT) scanner 202. The OCT scanner 202 is connected to the electrical connection within the cable 179 which in turn is connected to the sub-assembly controller 188. The OCT scanner 202 may be activated by the sub-assembly controller 188 to scan the patient's eye and assist in preparing an OCT image as is known in the art. In certain embodiments, if the OCT add-on 200 is active, the OCT scanner 202 may also be able to detect thickness of accumulated water, or other liquids, on the surface of the eye.

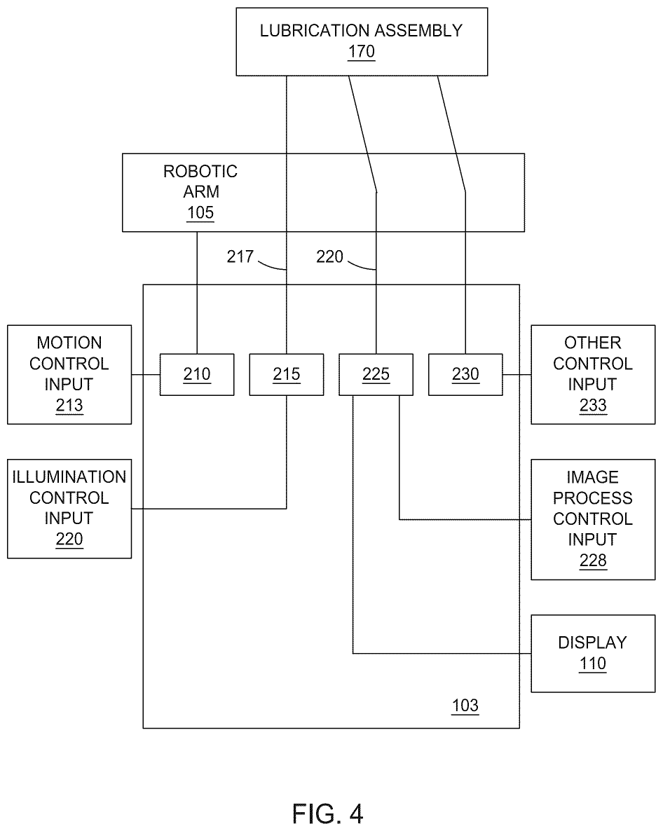

FIG. 4 is a block diagram schematically illustrating various components of the system 100 of FIG. 1. In the example of FIGS. 1, the cart 103 provides the base and physical support for the robotic arm 105, which in turn carries the lubrication assembly 170.

The cart 103 further includes a motion controller 210, which is operable to monitor and control movement of the various links of the robotic arm 105 to move the robotic arm 105 and to compute its position and the positions of its various joints and links, as well as that of the lubrication assembly 170. The motion controller 210 may receive control inputs from users of the system 100 via a motion control input device 213. During surgical procedures, the motion controller 210 is operable to move the robotic arm 105 so as to position and orientate the lubrication clement 175 of the lubrication assembly 170 so that may be disposed above a desired location of the outer surface of the patient's eye, so that a distal end of the lubrication element 175 may emit or deposit lubrication directly to the desired location.

The motion control input device 213 may include hand controls such as joysticks or keyboards, foot pedals, voice controls, or other suitable apparatus, which can be pre-programmed to control actuation of the robotic arm 105 via various motions and procedures therewith. Some robotic arms 105 may include sufficient internal feedback devices, such as internal force torque (FT) sensors, so that those robotic arms 105 can be moved as desired by direct movements applied manually to the robotic arms or their corresponding elements by users. Some robotic arms may be provided with force torque sensors operable to respond to the detection of forces or torques applied to elements of the robotic arm above predetermined acceptable limits. Such configurations can help to prevent damage to the patient's eye by preventing unintentional contact between the lubrication assembly 170 and the patient's eye.

The cart 103 may also include an illumination source 215. The illumination source 215 can be optically coupled with the illumination element 192, to deliver illumination energy from the illumination source 215 to the illumination clement 192 at the working end 172 of the lubrication assembly 170.

The illumination source 215 may typically be optically coupled to the illumination clement 192 via one or more optical delivery fibers 217. Illumination energy may in some embodiments be delivered to the illumination clement 192 via optical delivery fibers disposed inside and along the length of the links of the robotic arm 105.

The illumination energy may often be in the form of visible light, but may also include near-infrared or ultraviolet energy, or other illumination energies appropriate to the visualization desired for a given application.

Delivery of illumination energy from the illumination source 215 may be controlled by an illumination control input apparatus 220 operable by the system's users. Those users may use the illumination control input apparatus 220 to adjust, for example, the intensity or type (frequency, etc.) of the illumination energy, as they find most useful in particular circumstances. In certain embodiments, the illumination control input apparatus 220 comprises a physical knob for manually tuning characteristics of the illumination energy, such as a physical knob on a hand controller, foot pedal, cart 103, the surgical console 102, or other suitable device in an operating environment. In certain embodiments, the illumination control input apparatus 220 comprises a digital knob for manually tuning characteristics of the illumination energy, such as a digital control mechanism on a display monitor 110, cart 103, or the surgical console 102.

Illumination energy projected from the illumination source 215 is reflected, from within the patient's eye, back to the camera 195 at the working end 172 of the lubrication assembly 170. That reflected energy may be transmitted along optical return fibers 220 (or the same optical fiber 217 for delivering the illumination energy) or other appropriate elements to image receiving and processing apparatus 225, which may be housed inside or at the cart 103 or surgical console 102. The image receiving and processing apparatus 225 may include, for example, an active-pixel sensor based on a complementary metal-oxide semiconductor (CMOS) sensor. The optical return fibers 220 may preferably be disposed at least partially inside the links of the robotic arm 105, and then further to connect the camera 195 to the image receiving and processing apparatus 225.

Image process control input 228 may frequently be present and usable to control processing of the signals before the images are displayed. These controls may be relatively simple. The brightness and contrast of the displayed images might be adjustable by the users, for example.

More complicated adjustment and processing might be used as well, and with various degrees of automation. Displayed images might be stabilized or rotated into orientations more useful to the surgeon. Images from the camera 195 might be overlaid or otherwise combined with images from other devices or sensors, including the surgical microscope or other devices used for ophthalmic procedures.

In certain embodiments, the cart 103 may also carry or house an additional apparatus 230 usable with other working elements within the lubrication assembly 170, for example the sub-assembly controller 188. This additional apparatus 230 could include, for example, pumps, tanks, piping, and supplies for delivering lubrication to the surgical site or other locations of interest via the lubrication assembly 170. In certain embodiments, the additional apparatus 230 may be associated with additional control input mechanisms 233 and may be automated or under direct or indirect control of the system's users.

In certain embodiments, performing an ophthalmic procedure using the system 100 comprises the surgeon 112 and/or the assistant 115 placing the lubrication assembly 170 in an appropriate position relative to the eye of the patient 107. In certain embodiments where the lubrication assembly 170 is coupled to or incorporated into the robotic arm 105, placement of the lubrication assembly 170 may be done through direct or remote control of the robotic arm 105 via the motion controller 210 and motion controller input 213, while in other embodiments the surgeon 112 or assistant 115 may manually articulate the robotic arm 105 into position by physically moving each of the various links of the robotic arm 105 by hand. Once the robotic arm 105 has been manipulated to put the lubrication assembly 170 into position, the robotic arm 105 may be locked into place so that the lubrication assembly 170 remains in a static position throughout the duration of the ophthalmic procedure. In certain other embodiments where the lubrication assembly 170 is coupled to or incorporated into a surgical microscope, placement of the lubrication assembly 170 may be done through direct or remote control of the surgical microscope via the motion controller 210 and motion controller input 213, while in other embodiments the surgeon 112 or assistant 115 may manually articulate the surgical microscope into position by physically moving the surgical microscope by hand. Similarly, once the surgical microscope has been manipulated to put the lubrication assembly 170 into position, the surgical microscope may be locked into place so that the lubrication assembly 170 remains in a static position throughout the duration of the ophthalmic procedure.

In certain embodiments, after the lubrication assembly 170 has been placed into a desired first position, the lubrication assembly 170 may be selectively adjusted during the ophthalmic procedure to move into a second or subsequent desired position and/or to maintain its relative position over the patient's eye by continuously tracking its relative movement using the camera 195 disposed on the working end 172 of the lubrication assembly 170. Digital signals corresponding to images captured by the camera 195 may be sent to the image receiving and processing apparatus 225 and to the displays 110 where image analysis software contained therein continuously compares the received digital signals to a digital signal corresponding to the original or correct desired position of the lubrication assembly 170. For example, when the image analysis software detects that the received digital signal begins to differentiate from the digital signal corresponding to the correct desired position past a predetermined threshold, the system 100 alerts the surgeon 112 or assistant 115 via an audible alarm or message displayed on the displays 110 who may then take corrective measures to ensure that the lubrication assembly 170 is placed back into its intended position, either manually or through remote or indirect actuation. In certain embodiments, the system 100 may automatically instruct the robotic arm 105 or the surgical microscope to move back into the correct desired position when a misalignment is detected without the input of either the surgeon 112 or the assistant 115.

In certain embodiments, dispensation of a lubricant from the lubrication assembly 170 may be initiated by direct input from the surgeon 112 or the assistant 115 via an input 20, for example via actuation of a foot pedal or any other analog or digital input means such as a button, switch, keyboard command, or the like, or through audible commands issued by the surgeon 112/assistant 115 using known voice command means.

In certain embodiments, dispensation of the lubricant from the lubrication assembly 170 is dictated by a predetermined or time-based schedule. For example, the surgical console 102 or the cart 103 may comprise a clock and related software means which allows the surgeon 112 or the assistant 115 to establish a time interval between each time the release valve is actuated in order to release a predetermined amount of lubricant from the lubricant assembly 170. The lubricant assembly 170 will continuously and automatically emit lubricant according to the determined time interval for the duration of the ophthalmic procedure. If the surgeon 112 or assistant 115 determines that either too much or too little lubricant is being applied, they may change or edit the time interval accordingly until an appropriate amount of lubricant is being applied to the patient's eye.

In certain embodiments, dispensation of the lubricant from the lubrication assembly 170 is automatically and autonomously performed according to an image processing protocol. For example, digital signals corresponding to images captured by the camera 195 may be sent to the image receiving and processing apparatus 225 and to the displays 110 where image analysis software contained therein continuously detects or monitors an amount of glare within the image, the amount of glare corresponding to a relative amount of moisture or lubrication on the surface of the patient's eye. In certain embodiments, when the detected level of glare drops below a predetermined threshold value, thereby indicating a low level of moisture or lubricant on the surface of the patient's eye, the image receiving and processing apparatus 225 sends a signal to the lubrication assembly 170 which dispenses or deposits a predetermined amount of lubricant from the lubrication assembly onto the patient's eye. Conversely, when a large amount of glare has been detected, thereby indicating that the patient's eye has a sufficient amount of lubrication, no signal is sent to the lubrication assembly 170 and therefore no additional lubricant is added to the patient's eye. In certain embodiments, the image receiving and processing apparatus 225 may continuously monitor the amount of glare within the received image data so that lubricant may be added in real-time to the patient's eye during the ophthalmic procedure, however in other embodiments, the image receiving and processing apparatus 225 periodically detects the amount of glare within the received image data according to a predetermined schedule or time interval so that lubricant is only potentially added during selected moments of the ophthalmic procedure.

In certain embodiments, the image receiving and processing apparatus 225, which continuously monitors the amount of glare within the received image data, may also simultaneously monitor or track the position of the lubrication assembly 170 relative to the patient's eye or portions thereof. For example, if one portion of the patient's eye is detected to have a low level of moisture, the image receiving and processing apparatus 225 may signal the robotic arm 105 to automatically move the lubrication assembly 170 to that specific location so that an adequate amount of lubricant may then be applied thereon. In certain other embodiments, the image receiving and processing apparatus 225 may signal the cart 103 to emit an audio or visual alarm indicating for the surgeon 112/assistant 115 to manually move the lubrication assembly 170 to another specific portion of the patient's eye which requires lubricant. In this manner, the relative position of the lubrication assembly 170 to the patient's eye may be autonomously or manually adjusted during the ophthalmic procedure so that lubricant may be dispensed to the portions of the patient's eye where it is always most needed.

Because the dispersion of lubricant from the lubrication assembly 170 is completely configurable and customizable, the surgeon can not only ensure that the patient's eye is properly lubricated during the procedure, but can also selectively decide the most optimal times that lubricant is dispersed.

The description above has shown, described, and pointed out various features and configurations as applied in various examples. It should be understood, though, that various omissions, substitutions, and changes in the form and details of the example devices can be made without departing from the spirit of the disclosure. It should be understood as well that various features of the type described here can be utilized in various combinations, with individual features included omitted as desired and appropriate. None of these feature should be regarded as required in any particular combination, unless the description clearly requires otherwise. As will be recognized, the elements and combinations described here can be embodied in various forms, some of which may not provide all of the features and benefits described in this disclosure, as some features can be used or practiced separately from others. The scope of protection must therefore be defined primarily by the appended claims rather than the foregoing description, and the scope of those claims must be read to include the full scope of equivalents to which those claims are rightfully and legally entitled.

1. A lubrication assembly for lubricating an eye of a patient during an ophthalmic procedure, the lubrication assembly comprising:
a mounting bar;
an output coupled to the mounting bar;
means for disposing the output into a desired position relative to the eye of the patient coupled to the mounting bar;
a lubrication source fluidly coupled to the output;
a cable communicating a lubricant between the lubrication source and the output; and
a camera coupled to the mounting bar.

2. The lubrication assembly of claim 1, wherein the means for disposing the output into a desired position relative to the eye of the patient comprises a robotic arm coupled to a motion controller operable to move the robotic arm to extend the output into the desired position relative to the eye of the patient.

3. The lubrication assembly of claim 2, wherein the output and the camera are disposed on a working end of the robotic arm.

4. The lubrication assembly of claim 1, wherein the means for disposing the output into a desired position relative to the eye of the patient coupled to the mounting bar comprises a surgical microscope.

5. The lubrication assembly of claim 1, further comprising an illumination element, wherein the illumination element is configured to illuminate the eye of the patient, and wherein the lubrication assembly is further configured so that illumination reflected from the eye of the patient is received by the camera.

6. The lubrication assembly of claim 1, further comprising an input means configured to disperse lubricant from the output upon actuation of the input means.

7. The lubrication assembly of claim 1, wherein the camera is coupled to an image receiving and processing apparatus configured to detect an amount of glare within a digital signal corresponding to an image captured by the camera.

8. The lubrication assembly of claim 1, wherein the output comprises at least one of a needle-less syringe, eye dropper, pipette, tubing, or nozzle configured to dispense fluid in a precise and controlled manner.

9. The lubrication assembly of claim 1, wherein the mounting bar comprises an add-on slot configured to selectively accommodate at least one removable add-on therein.

10. The lubrication assembly of claim 9, wherein the at least one removable add-on comprises a corneal cross-link add-on, and wherein the corneal cross-link add-on comprises a UV light source and a syringe configured to disperse liquid vitamins or other fluids for facilitating corneal cross-linking.

11. The lubrication assembly of claim 9, wherein the removable add-on comprises an optical coherence tomography (OCT) scanner.

12. A method for lubricating an eye of a patient during an ophthalmic procedure comprising:
moving a lubrication assembly into a desired position relative to the eye of the patient;
selectively dispersing a lubricant from the lubrication assembly onto the desired position of the eye of the patient; and
monitoring a relative lubrication level of the eye of the patient for a duration of the ophthalmic procedure.

13. The method of claim 12, wherein moving the lubrication assembly into the desired position relative to the eye of the patient comprises actuating a motion controller operable to move a robotic arm comprising the lubrication assembly into the desired position.

14. The method of claim 12, wherein moving the lubrication assembly into the desired position relative to the eye of the patient comprises actuating a surgical microscope comprising the lubrication assembly into the desired position.

15. The method of claim 12, wherein selectively dispersing the lubricant from the lubrication assembly onto the desired position of the eye of the patient comprises at least one of actuating a foot pedal, actuating a button or switch, or issuing a voice command.

16. The method of claim 12, wherein selectively dispersing the lubricant from the lubrication assembly onto the desired position of the eye of the patient comprises dispersing the lubricant from the lubrication assembly according to a predetermined schedule or time interval.

17. The method of claim 12, wherein selectively dispersing the lubricant from the lubrication assembly onto the desired position of the eye of the patient comprises dispersing the lubricant from the lubrication assembly when the relative lubrication level of the eye of the patient is determined to be below a predetermined threshold.

18. The method of claim 12, wherein monitoring the relative lubrication level of the eye of the patient for the duration of the ophthalmic procedure comprises capturing a digital signal corresponding to an image of the patient's eye by a camera disposed on the lubrication assembly.

19. The method of claim 18, wherein selectively dispersing the lubricant from the lubrication assembly onto the desired position of the eye of the patient comprises detecting an amount of glare within the digital signal corresponding to an image of the patient's eye and dispersing the lubricant from the lubrication assembly when the detected amount of glare is determined to be below a predetermined threshold.

20. The method of claim 19, wherein detecting the amount of glare within the digital signal corresponding to an image of the patient's eye and dispersing the lubricant from the lubrication assembly comprises automatically and autonomously processing the digital signal, at an image receiving and processing apparatus, to detect the amount of glare within the digital signal and automatically and autonomously dispersing the lubricant from the lubrication assembly when the detected amount of glare is determined to be below a predetermined threshold.
